(19) United States
(12) Patent Application Publication
(10) Pub. No.: US 2014/0377861 A1
(43) Pub. Date: Dec. 25, 2014

(54) Precise Control of Recombinant Protein Levels by Engineering Translation

(71) Applicant: The Board Trustees of the Leland Stanford Junior University, Palo Alto, CA (US)

(72) Inventors: Clifford Lee Wang, Redwood City, CA (US); Joshua Paul Ferreira, Stanford, CA (US)

(21) Appl. No.: 14/371,125

(22) PCT Filed: Jan. 11, 2013
(86) PCT No.: PCT/US2013/021225

§ 371 Date: Jul. 8, 2014

Related U.S. Application Data
(60) Provisional application No. 61/586,005, filed on Jan. 12, 2012.

(51) Int. Cl. C12N 15/10

(52) U.S. Cl. CPC C12N 15/1086
USPC 435/366; 506/16; 506/2; 435/320.1

(57) ABSTRACT

Compositions and methods are provided for user-specified fine-tuned protein expression levels, by controlling the initiation of protein translation; and a model for analysis of expression. This method of control can be used to vary, tune, and optimize protein production in genetically engineered organisms, cells, or devices.

### GOVERNMENT RIGHTS

This invention was made with Government support under contract 0846392 awarded by the National Science Foundation. The Government has certain rights in this invention.

### BACKGROUND

The study of gene function often requires changing the expression of a gene and evaluating the consequences. In such methods, for various purposes it is desirable to have a closely defined level of protein activity. For example, applications such as metabolic optimization and control analysis necessitate a continuous set of expression levels with only slight increments in strength to cover a specific window around the wild-type expression level of the studied gene.

One approach to this need has been to utilize promoters of different strengths. Upstream from a structural gene encoding a polypeptide of interest there is a DNA sequence region (normally referred to as the promoter region) to which RNA polymerase and other transcription factors bind. The RNA polymerase catalyzes the assembly of the mRNA complementary to the appropriate DNA strand of the polypeptide coding region. Most “promoter regions” comprise a RNA polymerase recognition site (often including TATA box) located upstream from the start of the coding region (structural gene) and the site for accurate initiation of transcription. Modification in the “promoter region” may result in enhanced transcription levels, which again may lead to increased expression and production yields. This approach generally consists of inserting a library of promoters in front of the gene to be studied, whereby the individual promoters might deviate either in their spacer sequences or bear slight deviations from a consensus promoter sequence. However there are drawbacks to this approach, and optimization generally requires a highly cell-specific analysis. Further, there are many instances in which high level expression is undesirable, and where a regulated low level of expression is required.

Methods of achieving stable expression at a desired target level are of great interest for research and therapeutic purposes. The present invention addresses this need.

Published documents include Gibson et al., Nat Methods 6 (5), 343 (2009); and Naviaux et al., J Virol 70 (8), 5701 (1996).

### SUMMARY OF THE INVENTION

Compositions and methods are provided for user-specified fine-tuned protein expression levels, by controlling the initiation of protein translation. This method of control can be used to vary, tune, and optimize protein production in genetically engineered organisms, cells, or devices. Because the relevant mechanisms of translation are highly conserved in eukaryotes, the methods of the invention are applicable to all eukaryotes, including humans, plants, and yeasts.

Conventional genetically engineered promoter systems that control expression level by modulating transcription typically are capable of a 20-40 fold range. With the methods of the invention, control of translation initiation by manipulating initiation sequences and or adding one or more upstream reading frames is capable of generating an expression in the 200-600 fold range. Because expression is controlled at the stage of translation, two genes can be expressed with expression levels independent of each other from the same mRNA transcript. In eukaryotes, this is not possible when a promoter-based control approach, since each promoter generates a single transcript. This advantage allows the GOI to be expressed at a level independent of an antibiotic selection gene or cellular marker.

In the methods and genetic constructs of the present invention the level of recombinant protein production in a system of interest, e.g. a cell, cell-free synthetic system, and the like, is specified by controlling the rate of translation initiation of a gene of interest (GOI). Generally the sequences controlling translation initiation are operably linked to a promoter, often a strong promoter, and to an open reading of the gene of interest. In some embodiments, stop codons in all three reading frames are inserted upstream of all sequences, including the regulatory upstream ORF and the GOI ORF.

The rate of translation initiation is controlled by one or both of (a) manipulating the nucleotide base sequences specifying translation initiation sites and (b) adding a regulatory short open reading frame upstream of the gene of interest, which may comprise at least two, at least three and not more than 10 codons, including the initiation and termination codons, and is generally located a minimal distance from the GOI ORF, e.g. at least about 2 and not more than about 10 nucleotides distance. Such a regulatory ORF decreases rates of translation initiation. To be able to specify the level of translation initiation of the GOI ORF, and thus protein production, with the greatest degree of control and predictability, the upstream ORF should not be in-frame with the GOI ORF, i.e. the number of bases between the upstream ORF stop codon and the start codon of the GOI ORF should not be zero or a multiple of three. The rate of translation initiation of the GOI ORF, and thus protein production, is specified by manipulating the initiation sequences of both the upstream ORF and the downstream GOI ORF. The length of the regulatory ORF can be varied to achieve different levels of GOI expression. More than one upstream regulatory ORF can be employed. An out-of-frame start codon can also be inserted shortly after the GOI's start codon. This can be helpful when controlling the expression level of proteins that have methionines within the amino acid sequence.

In some embodiments of the invention a library of expression constructs is provided, where the library comprises a plurality of translation initiation sequences, which optionally include one or more upstream regulatory ORFs, as described herein. In some embodiments the expression of a GOI is screened by insertion into a library of expression constructs, and introducing the library into a cell culture, animal or other organism, or cell-free synthetic system. Combined with single-cell analysis methods such as flow cytometry, gene/protein dose response experiments are performed. A cell or expression system having the desired expression level may be selected for further expansion.

Antibiotic resistance genes or other cellular marker or reporter genes can be expressed independently using an internal ribosome entry site (IRES) downstream of the GOI. These genes downstream of the IRES can also be controlled using the translation initiation control method. Modified translation initiation sequences and upstream ORFs can be inserted in a targeted manner to generate transgenic cells, animals and plants. In this way, the expression of endogenous genes (as opposed to ectopic genes) can be manipulated.

Using high efficiency gene targeting technologies, for example directed zinc finger nucleases or TAL nucleases, translation initiation sequences and upstream ORFs can be used to replace initiation sequences in patients or patient cells that are later transplanted into the patient. As a gene therapy tool, the invention can be used to treat patients where an aberrant level of expression is part of the pathology of a patient ailment.

A mathematical model that allows prediction and design of desired translation initiation sequences is also provided. In some embodiments of the invention a method for synthesizing a protein of interest at a desired expression level is provided, where the method comprises inputting a translation initiation sequence into the provided model, determining the predicted level of expression, and generating a DNA construct comprising the translation initiation sequence, which is optionally operably linked to coding sequence for the protein of interest.

### BRIEF DESCRIPTION OF THE DRAWINGS

FIG. 1. Synthetic uORFs specify and tune expression levels. (A) Schematic of engineered mRNA transcripts. GFP, green fluorescent protein; NNN_{G}, bases preceding the GFP ORF; uORF, upstream open reading frame with sequence AUGGGCUGA where AUG and UGA are start and stop codons, respectively; NNNᵤ, bases preceding uORF; (NNN)ₛ, non-AUG start codon; IRES, internal ribosome entry site; RFP, red fluorescent protein; 5′m, 5′ RNA cap; AAA, poly-A tail. Non-N bases represent exact bases preceding the ORFs. All ORFs shown contain a G at the +4 position. (B-F) GFP expression in PD31 cells. (B) Effect of different initiation sequences without use of uORFs (construct 1). The often employed sequence GCCACCAUGG (positions −6 to +4) was utilized by one construct, represented by GCCACC. (C) Effect of different numbers of uORFs (constructs 1-4). (D) Effect of distance (n) between upstream and GFP ORFs, where n is the number of bases after the uORF stop codon and before the GFP start codon (construct 5). (E) Variation of the 3 bases preceding the uORF and GFP ORF (construct 6) where GFP employed an AUG start codon. (F) Use of non-AUG start codons (in parentheses) to express GFP, where uORFs and bases preceding uORFs were varied (constructs 7 and 8). (G) Expression in different cell lines. One construct, RFP only, contained no GFP gene. Translation level is reported as GFP fluorescence intensity normalized to RFP fluorescence intensity. Except for the transiently-transfected 293 cells in panel G, all expression constructs were stably integrated into the genome.

FIG. 2. Effect of uORFs on protein translation described by a leaky initiation model. (A) Schematic of leaky initiation mechanism. Ribosome, blue double oval; 5′m, 5′ RNA cap; AAA, poly-A tail, gene of interest (GOI). In experiments, the GOI was GFP. Arrows indicates flow of ribosomes. (B) Model prediction based on a leaky initiation mechanism plotted against experimentally observed GFP translation levels. If model and predict agreed perfectly, data points would fall on the dotted line. The R² correlation value was 0.92.

FIG. 3. p21 dose-response assessed by employing initiation sequences with uORFs. (A) Expression of p21 fused to blue fluorescent protein and an estrogen receptor domain (p21-BFP-ER) in wild-type or p21-deficient (−/−) HCT-116 cells. Activation by addition of 4-OHT. (B) Immunoblot with anti-p21 and anti-pRB antibodies. IR, cells exposed to ionizing radiation. (C) Cell-cycle population distribution at different p21-BFP-ER levels specified using different initiation sequences and synthetic uORFs. Cells were induced with 4-OHT for 24 hours.

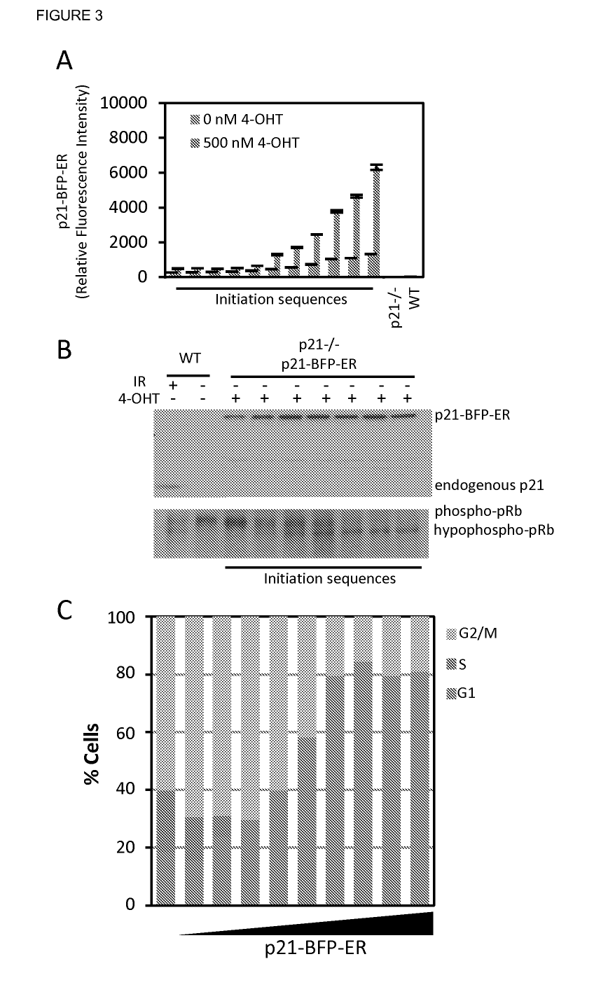

FIG. 4. Schematic of leaky translation model. uORFs reduce the flux of ribosomes that reach the downstream primary ORF. methylated 5′ RNA cap, 5′m; ribosomal subunits and complexes, blue ovals; ORFs, rectangles; polyA tail of mRNA, AAA

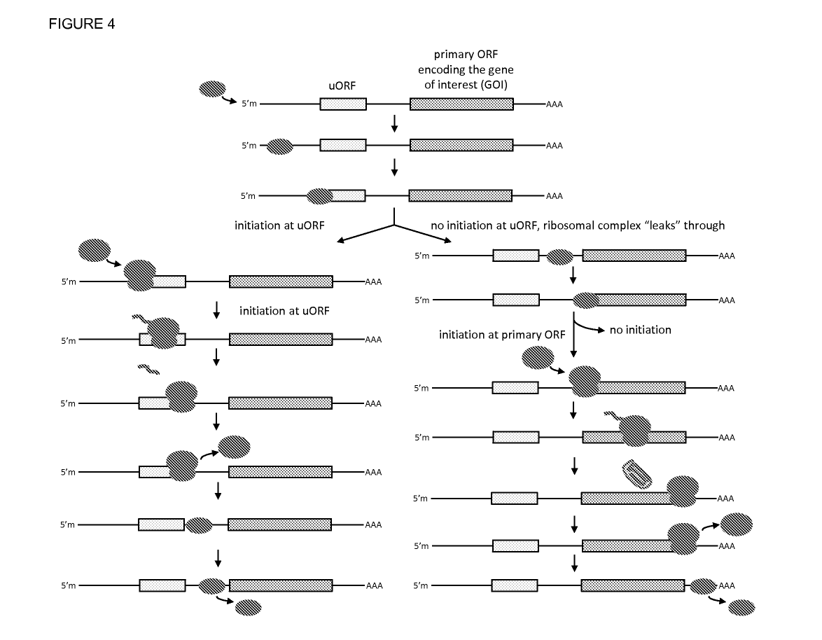

FIG. 5. Mathematical model predicts expression, as described in Example 2. Equations describe probability based decisions involved in expression of the gene of interest, which in our experiments was GFP. GFP expression (G) vs the strength of translation initiation sequence of the upstream open reading frame (SU) and the strength of the translation initiation sequence of the GFP gene (SG). Experimental results closely fit the mathematical model (red wire frame surface)

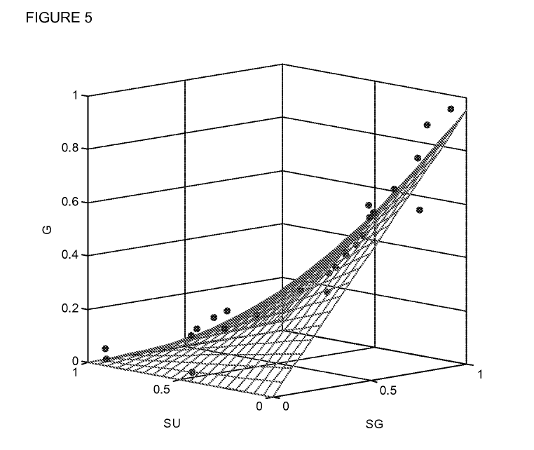

FIG. 6. Independent bi-cistronic expression scheme. Varying expression by engineering translation initiation sequences and upstream open reading frames allows expression control of a gene of interest (here, GFP) without affecting expression of a second gene, e.g. antibiotic resistance gene such as puromycin resistance (PuroR) on the same mRNA transcript. Full expression control is achieved by specifying the three bases (NNN) preceding the start codon (AUG) of both a regulatory, upstream 2-amino acid ORF and a downstream gene of interest, here green fluorescent protein (GFP). Retroviral long terminal repeat (LTR), internal ribosomal entry site (IRES), stop codon (TGA).

### DETAILED DESCRIPTION OF THE EMBODIMENTS

The present application refers to various patents, publications, books, articles, and other references. The contents of all of these items are hereby incorporated by reference in their entirety.

### I. Definitions

To facilitate understanding of the invention, the following definitions are provided. It is to be understood that, in general, terms not otherwise defined are to be given their meaning or meanings as generally accepted in the art.

The invention relates to a DNA sequences for regulating expression of a structural gene encoding a polypeptide in a eukaryotic host cell comprising (a) a first DNA sequence which comprises a translation initiation site; and may further comprise (b) one or more DNA sequence(s) providing a regulatory short open reading frame upstream of the gene of interest, which may comprise at least two, at least three and not more than 10 codons, including the initiation and termination codons, and is generally located a minimal distance from the GOI ORF, e.g. at least about 2 and not more than about 10 nucleotides distance. Such a regulatory ORF decreases rates of translation initiation. The upstream ORF is generally not in-frame with the GOI ORF. The rate of translation initiation of the GOI ORF, and thus protein production, is specified by manipulating the initiation sequences of both the upstream ORF and the downstream GOI ORF. The length of the regulatory ORF can be varied to achieve different levels of GOI expression. More than one upstream regulatory ORF can be employed. The invention also relates to a DNA construct and an expression vector and a host cell comprising the DNA sequence of the invention.

An ORF is defined as a sequence with a base length that is a multiple of three, starts with AUG, NUG, ANG, or AUN (where N is A, C, U or G), and ends with a stop codon (TAA, TGA, or TAA)

While translation initiation occurs on an RNA molecule, initiation sites are generally manipulated by engineering and specifying the bases at the DNA level, which are then transcribed into RNA. For example, the seven base initiation sequence TTTAUGG in a messenger RNA is achieved by using the DNA sequence TTTATGG at the start of the ORF of the GOI. Decreased rates of translation initiation are achieved by using non-AUG start codons such as ACG or CUG.

The terms “DNA sequence” and “nucleic acids sequence” may be used interchangeably.

The term “operably linked” is defined herein as a configuration in which, e.g., a DNA sequence of the invention is appropriately placed at a position relative to a polypeptide coding DNA sequence such that regulated transcription levels are obtained.

“Coding sequence” is defined herein as a nucleic acid or DNA sequence, which is transcribed into mRNA and translated into a polypeptide when placed under the control of the appropriate control sequences. The boundaries of the coding sequence are generally determined by a ribosome binding site located just upstream of the open reading frame at the 5′ end of the mRNA and a transcription terminator sequence located just downstream of the open reading frame at the 3′ end of the mRNA. A coding sequence can include, but is not limited to, genomic DNA, cDNA, semi-synthetic, synthetic, and recombinant nucleic acid sequences.

“Nucleic acid construct” or “DNA construct” is defined herein as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term expression cassette when the nucleic acid construct contains all the controlling sequences required for expression of a coding sequence.

A “Kozak” consensus sequence is a sequence that occurs on eukaryotic mRNA and has the consensus (gcc)gccRccAUGG, where R is a purine (adenine or guanine) three bases upstream of the start codon (AUG), which is followed by another ‘G’. The Kozak consensus sequence plays a major role in the initiation of the translation process. This sequence on an mRNA molecule is recognized by the ribosome as the translational start site. The ribosome requires this sequence, or a variant thereof to initiate translation.

The Kozak site varies on different mRNAs and the amount of protein synthesized from a given mRNA is dependent on the strength of the Kozak sequence. (see Kozak (1984) Nature 308 (5956):241-246).

Some nucleotides in this sequence are more important than others: the AUG is most important because it is the actual initiation codon encoding a methionine amino acid at the N-terminus of the protein. Numbering the A of the AUG start codon as +1, the most important rate-determining bases of translation initiation are those from −3 to +4. For even finer tuning, bases −6 to −4 also affect the rate of translation initiation, though less than those from −3 to +4.

At the start of translation a pre-initiation complex (43S subunit, or the 40S and tRNA) accompanied by protein factors move along the mRNA chain towards its 3′-end, scanning for a start codon on the mRNA. The Met-charged initiator tRNA is brought to the P-site of the small ribosomal subunit by eukaryotic Initiation Factor 2 (eIF2). It hydrolyzes GTP, and signals for the dissociation of several factors from the small ribosomal subunit which results in the association of the large subunit (or the 60S subunit). The complete ribosome (80S) then commences translation elongation, during which the sequence between the ‘start’ and ‘stop’ codons is translated from mRNA into an amino acid sequence.

The term “recombinant” expression or production means in the context of the present invention that the polypeptide in question is expressed from a gene exogenous to the donor cell, that a DNA construct comprising the gene encoding the polypeptide in question is introduced into a cell and expressed from this genetically modified cell; or that a genetically modified translation initiation sequence, optionally including a regulatory upstream ORF, is introduced 5′ to an endogenous gene.

The parts constituting the DNA sequence of the invention or the whole DNA sequence of the invention may be artificial or may be derived from a eukaryotic organism.

The structural gene may encode any polypeptide. In an embodiment the structural gene encodes a polypeptide with a biological activity. In a some embodiments the structural gene encodes a polypeptide exhibiting enzymatic activity. In other embodiments the polypeptide is a ligand, a receptor, a structural protein, and the like as known in the art.

The invention also relates to a DNA construct comprising a DNA sequence of the invention for regulating transcription. The DNA construct of the invention is operative in a eukaryotic host cell and the DNA sequences of the invention are operable linked with a structural gene encoding a polypeptide and a terminator.

The invention also relates to an expression vector comprising a DNA construct of the invention. The DNA construct may further comprise a signal peptide coding region. In such embodiment the transcribed and expressed polypeptide will be secreted. An expression vector of the invention may comprise a DNA construct of the invention wherein the DNA sequence of the invention is operably linked to a single copy of a structural gene encoding a polypeptide, and optionally leader sequence located upstream of the structural gene encoding the polypeptide.

Control sequences include, but are not limited to, a leader, a polyadenylation sequence, a propeptide sequence, a promoter or part thereof, a signal sequence, and a transcription terminator. The control sequences may be provided with linkers for the purpose of introducing specific restriction sites facilitating ligation of a nucleic acid sequence encoding the polypeptide in question which is operably linked to a control element of the invention.

The DNA sequence of the invention may comprise a promoter, a mutant thereof, or a truncated promoter or a hybrid promoter. The promoter may be any nucleic acid sequence, which shows transcriptional activity in a eukaryotic host cell of choice and may be obtained from genes encoding extracellular or intracellular polypeptides either homologous or heterologous to the host cell. Each promoter sequence may be native or foreign to the nucleic acid sequence encoding the polypeptide (structural gene) and native or foreign to the eukaryotic host cell in question. Each control sequence may be native or foreign to structural gene encoding the polypeptide in question to the transcribed and expression.

Promoters have a complex block-modular structure and contain numerous short functional elements such as a transcription factor binding site, a RNA polymerase recognition site, a mRNA initiation site. These sequences have no exact uniform location and are dispersed in the 5′-flanking region up to about 1 kb upstream of the mRNA initiation site where transcription starts.

The present invention also relates to recombinant expression vectors comprising a DNA sequence or DNA construct of the invention for regulating transcription, and transcriptional and translational stop signals. The various DNA and control sequences described above may be joined together to produce a recombinant expression vector, which may include one or more convenient restriction sites to allow for insertion or substitution of the nucleic acid sequence encoding the polypeptide at such sites. Alternatively, the structural gene encoding a polypeptide may be expressed by inserting the DNA sequence of the invention or a DNA construct into an appropriate vector for expression. In creating the expression vector, the polypeptide coding sequence is located in the vector so that the coding sequence is operably linked with the appropriate control sequences for expression, and possibly secretion.

The recombinant expression vector may be any vector (e.g., a plasmid or virus), which can be conveniently subjected to recombinant DNA procedures and can bring about the expression of the structural gene encoding the polypeptide. The choice of the vector will typically depend on the compatibility of the vector with the eukaryotic host cell into which the vector is to be introduced. The vectors may be linear or closed circular plasmids. The vector may be an autonomously replicating vector, i.e., a vector which exists as an extrachromosomal entity, the replication of which is independent of chromosomal replication, e.g., a plasmid, an extrachromosomal element, a minichromosome, a cosmid or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total DNA to be introduced into the genome of the host cell, or a transposon.

The vectors of the present invention may contain an element(s) that permits stable integration of the vector into the host cell genome or autonomous replication of the vector in the cell independent of the genome of the cell.

The vectors of the present invention may be integrated into the host cell genome when introduced into a host cell. For integration, the vector may rely on the nucleic acid sequence encoding the polypeptide or any other element of the vector for stable integration of the vector into the genome by homologous or none homologous recombination. Alternatively, the vector may contain additional nucleic acid sequences for directing integration by homologous recombination into the genome of the host cell.

For autonomous replication, the vector may further comprise an origin of replication enabling the vector to replicate autonomously in the host cell in question. Examples of bacterial origins of replication that are, for example, useful in the initial generation of the vectors are the origins of replication of plasmids pBR322, pUC19, pACYC177, pACYC184, pUB110, pE194, pTA1060, and pAMβ1. Examples of origin of replications for use in a yeast host cell are the 2 micron origin of replication, the combination of CEN6 and ARS4, and the combination of CEN3 and ARS1. The SV40 replication origin is useful in mammalian cells. The origin of replication may be one having a mutation which makes its functioning temperature-sensitive in the host cell (see, e.g., Ehrlich, 1978, Proceedings of the National Academy of Sciences USA 75:1433).

The invention also relates to eukaryotic host cell comprising a DNA sequence of the invention for regulating transcription or a DNA construct of the invention or an expression vector of the invention. The eukaryotic host cell of the invention comprises a structural gene encoding a polypeptide. The term “host cell” encompasses any progeny of a parent cell, which is not identical to the parent cell due to mutations that occur during replication. The cell is preferably transformed with a vector comprising a DNA sequence for regulating transcription of the invention operably linked to a structural gene followed, in particular by integration of the vector into the host chromosome.

The host cell is usually a eukaryote, such as a mammalian cell, an insect cell, a plant cell or a fungal cell.

### METHODS OF THE INVENTION

Compositions and methods are provided for user-specified fine-tuned protein expression levels, by controlling the initiation of protein translation. This method of control can be used to vary, tune, and optimize protein production in genetically engineered organisms, cells, or devices. Because the relevant mechanisms of translation are highly conserved in eukaryotes, the methods of the invention are applicable to all eukaryotes, including humans, plants, and yeasts.

In the methods and genetic constructs of the present invention the level of recombinant protein production in a system of interest, e.g. a cell, cell-free synthetic system, and the like, is specified by controlling the rate of translation initiation of a gene of interest (GOI). Generally the sequences controlling translation initiation are operably linked to a promoter, often a strong promoter, and to an open reading of the gene of interest. In some embodiments, stop codons in all three reading frames are inserted upstream of all sequences, including the regulatory upstream ORF and the GOI ORF.

In some embodiments the sequence of the region upstream of the gene of interest comprises a regulatory ORF of at least two and not more than 10 codons in length, which regulatory ORF is from 2 to 10 nucleotides distant from the initiation codon of the GOI. The translation initiation sequence upstream of the regulatory ORF is genetically manipulated to adjust the level of expression, where a strong signal for the regulatory ORF results in decreased expression from the GOI. In some embodiments, the region upstream of the GOI is selected from the sequences set forth in Table 1.

In some embodiments a library comprising a plurality of upstream regulatory sequences is generated in which a regulatory ORF of at least two and not more than 10 codons in length, which regulatory ORF is from 2 to 10 nucleotides distant from the initiation codon of the GOI. The translation initiation sequences upstream of the regulatory ORF are varied adjust the level of expression. A library of such regulatory sequence may comprise 3, 5, 7, 10, 12, 15, 17, 20 or more different regulatory sequences, which produce a variation in expression of a linked gene of interest of at least 100 fold range. In some embodiments the range of expression is at least 200-fold, at least 300-fold, at least 400-fold, at least 500-fold or more.

The library of regulatory sequences is useful for screening to select the regulatory sequence that provides a desired for level of expression. For such screening purposes the regulatory sequence may be provided in a genetic construct, e.g. a plasmid, retrovirus, etc. The gene of interest is operably linked to the regulatory sequence. For screening purposes the genetic construct is introduced into a system for expression, e.g. a cell, transgenic animal, cell-free expression system, and the like. The level of expression is determined by any convenient method and will be selected based on the gene of interest, e.g. by blotting, RIA, functional assay, flow cytometry staining for the protein of interest, and the like as known in the art. A construct selected for providing the appropriate level of expression may be expanded for the desired purpose.

In one aspect the invention relates to a method of producing a polypeptide, comprising: (a) cultivating a host cell harboring a gene of interest under control of a regulatory sequence of the invention, in a nutrient medium suitable for production of the polypeptide; and (b) recovering the polypeptide from the nutrient medium. The host cell may be any of the above mentioned. The regulatory sequence of the invention is located upstream to a gene of interest encoding a polypeptide, which may be native or foreign to the host cell.

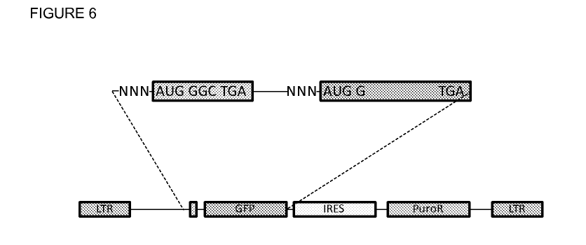

In some specific embodiments of the invention, a genetic construct is provided as set forth in FIG. 6. Varying expression by engineering translation initiation sequences and upstream open reading frames allows expression control of a gene of interest without affecting expression of a second gene, e.g. antibiotic resistance gene on the same mRNA transcript. Full expression control is achieved by specifying the three bases (NNN) preceding the start codon (AUG) of both a regulatory, upstream 2-amino acid ORF and a downstream gene of interest. From the same transcript a GOI can be expressed at a low level while the antibiotic selection gene is expressed at a high level.

The inventive composition comprising the regulatory sequences of the invention operably linked to a gene of interest may be used as a gene therapy agent for preventing and treating various hereditary diseases.

The composition for gene therapy of the present invention may further comprise pharmaceutically acceptable carriers. Any of the conventional procedures in the pharmaceutical field may be used to prepare oral formulations such as tablets, capsules, pills, granules, suspensions and solutions; rejection formulations such as solutions, suspensions, or dried powders that may be mixed with distilled water before injection; locally-applicable formulations such as ointments, creams and lotions; and other formulations.

Carriers generally used in the pharmaceutical field may be employed in the composition of the present invention. For example, orally-administered formulations may include binders, emulsifiers, disintegrating agents, excipients, solubilizing agents, dispersing agents, stabilizing agents, suspending agents, coloring agents. Injection formulations may comprise preservatives, solubilizing agents or stabilizing agents. Preparation for local administration may contain bases, excipients, lubricants or preservatives. Any of the suitable formulations known in the art (Remington's Pharmaceutical Science [the new edition], Mack Publishing Company, Eaton Pa.) may be used in the present invention.

The inventive composition may be administered orally or via parenteral routes such as intravenous, intramuscular, subcutaneous, intra-abdominal, sternal and arterial injection or infusion, or topically through rectal, intranasal, inhalational or intraocular administration.

The typical daily dose of the active ingredient may range from 0.001 to 5 mg/kg body weight, preferably from 0.01 to 0.5 mg/kg body weight and can be administrated in a single dose or in divided dose. However, it should be understood that the amount of the effective ingredient actually administrated ought to be determined in light of various relevant factors including the conditions to be treated, the chosen route of administration, the age, sex and body weight of the individual patient, and the severity of the patient's symptom. Therefore, the above dose should not be construed as a construed as a limitation to the scope of the invention in any way.

Also provided is a mathematical model that predicts the behavior of genes regulated by upstream open reading frames, including without limitation the regulatory sequences of the present invention. The model is set forth in detail in Example 2 herein. The prediction model can be used to predict protein expression from, for example, sequenced genes and genomes.

The analysis and prediction model can be implemented in hardware or software, or a combination of both. In one embodiment of the invention, a machine-readable storage medium is provided, the medium comprising a data storage material encoded with machine readable data which, when using a machine programmed with instructions for implementing the algorithm, provides for a method of predicting the behavior of genes regulated by upstream open reading frames.

A machine configured to implement the algorithm provided herein can be used for a variety of purposes involved with testing and predicting expression of genes. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. “Media” refers to a manufacture that contains the mathematical information of the present invention. The model and instructions of the present invention can be recorded on computer readable media, e.g. any medium that can be read and used to configure a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

The following examples are intended to further illustrate the present invention without limiting its scope.

### EXPERIMENTAL

Vector construction. Sequences to modify translation initiation were added to monomeric enhanced green fluorescent protein (EGFP A207K, hereafter referred to as GFP) by PCR amplification. GFP was amplified using varied forward primers and the reverse primer, 5′-CGGAATTGGCCGCCCTAGATGCATGCTTA TTCGAACTTGTACAGCTCGTCC ATGCCGA-3′ and then inserted into the retroviral expression plasmid pCru5-IRES-mCherry at the XhoI and SphI restriction sites using a previously described DNA assembly method.

Cell culture. PD-31 cells were cultured in RPMI-1640 medium with fetal bovine serum (FBS), 2 mM glutamine, 1 mM sodium pyruvate, and 0.05 mM 2-mercaptoethanol. K562 cells were cultured in RPMI-1640 with FBS and 2 mM glutamine. HEK-293T cells were cultured in Dulbecco's Modified Eagle Medium (DMEM) with FBS, 4.5 g/ml glucose and 2 mM glutamine. All cells were cultured at 37° C. with 5% CO₂.

To evaluate the engineered initiation sequences by transient transfection, the retroviral expression vectors were introduced to HEK-293T cells using the calcium phosphate precipitation method (CalPhos Mammalian Transfection Kit, Clontech Laboratories, Inc.).

To evaluate the engineered initiation sequences in stable cell lines, PD31 and K562 cells were transduced with the retroviral vectors. Retroviral particles were produced by co-transfecting the retroviral expression vectors with either pCL-Eco (ecotropic pseudotyping for PD31) or pCL-Ampho (amphotropic pseudotyping for K562) using the calcium phosphate precipitation method. Virus-containing supernatant was harvested and added with 3 μg/ml polybrene (hexadimethrine bromide) to cells. Virus was titered so that transduced cells received a single copy of the vectors.

Flow cytometry. The GFP and the red fluorescent protein mCherry (hereafter referred to as RFP) expression were quantified by measuring fluorescence intensities by flow cytometry. Cells were analyzed on a LSRII flow cytometer (BD Biosciences, Franklin Lakes, N.J., USA). Flow cytometry data was first analyzed with FlowJo software (Tree Star, Ashland, Oreg., USA). The rate of translation initiation was gauged by computing the quotient of GFP to RFP levels.

### Results

Our goal was to control gene expression by specifying the level of translation initiation of a gene of interest (GOI). To achieve this goal, we added nucleotide sequences 5′ to the open reading frame (ORF) of the GOI (Table 1) and investigated four strategies, we (1) varied the bases adjacent to the start codon of the GOI (primarily bases at positions −3, −2, and −1, but also −6, −5, −4, and +4, where position +1 is the first base of the open reading frame (ORF) of the GOI), (2) added a short ORF (2 amino acids in most cases) upstream of the GOI, (3) varied the three bases preceding the start codon of the upstream ORF, (4) varied the distance between the upstream ORF and downstream GOI, and (5) used different start codons, including AUG, ACG, and TTT.

We first evaluated expression by transient transfection of HEK-293T cells with vectors where the GOI was GFP, and RFP was expressed using a downstream internal ribosome entry site. Flow cytometry was then used to measure the levels of GFP and RFP; because RFP expression was found to be independent of the expression level of the GFP, here we have chosen to report the level of translation initiation as expression of GFP per mRNA transcript, computed as the quotient GFP/RFP.

Varying the bases at or adjacent to the start codon of GFP led to varying levels of GFP expression. The strong, consensus translation initiation sites described by Kozak (GCCACCAUGG, CACCAUGG, GCCAUGG, ACCAUGG)—produced the highest levels of expression. Some sequences that varied from the consensus (GAAAUGG, GUUAUGG, GGGAUGG) also produced high levels of expression, while others that varied from the consensus (CAG, CCC, UAA, UCC, UCGAUGG, CUUAUGG, UAGAUGG, CGAAUGG, CGGAUGG, UUGAUGG, UUUAUGG) produced levels between 50-90% of that of the Kozak consensus sequences. However, although varying the bases in the initiation site did change the expression level of GFP, we were not able to effectively specify a full range of expression levels.

Next, we introduced a two-amino acid ORF with a strong initiation site (ACCAUGG) 8 bases upstream of GFP. This led to an 85% suppression of GFP that itself was equipped with the strong initiation site ACCAUGG. We hypothesized that varying the distance between the upstream ORF and the GFP's ORF would vary the effect of the upstream ORF on GFP expression. Yet we found little difference in expression when this distance ranged from 5 to12 bases.

We next hypothesized that decreasing the strength of the translation initiation site of the upstream ORF would lessen the suppression of GFP expression. Indeed this was found to be the case; for example, a weaker upstream initiation site, UUU, led to only 45% suppression of GFP (when the GFP was equipped with the strong initiation site ACCAUGG). In general we found that the strength of initiation at the upstream ORF inversely affected the expression level of GFP. By varying the strengths of the initiation sites of the upstream ORF and the downstream GFP, we were able to produce a full range of expression levels. We also found that, instead of AUG, ACG or TTT could be used as start codons for GFP to produce significantly reduced levels of expression. By combining various strategies to affect translation initiation, we were able to generate expression over a 260-fold range in transiently transfected HEK-293 cells.

The same vectors employing the different translation initiation sequences were also used to generate stably transduced cell lines. The relative order of expression levels from the various constructs in the stably transduced PD-31 and K562 cells were nearly identical to that of the transiently transfected HEK-293 cells. This suggests that there may not be many cell-type specific factors involved in translation initiation—i.e., the translation machinery is relatively conserved between different cells and tissues. The range of expression achieved by the various constructs did vary between the cell lines though. The achievable expression range was 290-fold in PD-31 cells and 620-fold in K562 cells.

TABLE 1
Engineered translation
initiation sequences

| # | mRNA sequence (5′ to 3′)* |
|---|---|
| 1.2 | ACC-AUGG |
| 1.3 | ACC-AUGA |
| 1.4 | UCC-AUGA |
| 1.7 | UUU-AUGA |
| 1.8 | UUU-UUUA |
| 1.9 | ACC.AUGUUUUGAUUU-AUGA |
| 1.11 | ACC.AUGUUUUGAU-AUGA |
| 1.12 | ACC.AUGUUUUGA-AUGA |
| 1.13 | ACC.AUGUUUUG-AUGA |
| 1.14 | ACC.AUGUUUU-AUGA |
| 1.15 | ACC.AUGUU-AUGA |
| 1.22 | ACC.AUGUUUUG-ACGA |
| 2.1 | CACC-AUGG |
| 2.3 | AUC-AUGG |
| 2.4 | ACU-AUGG |
| 2.5 | AUU-AUGG |
| 2.6 | CCC-AUGG |
| 2.7 | GCC-AUGG |
| 2.8 | UCC-AUGG |
| 2.9 | GAU-AUGG |
| 2.10 | UGA-AUGG |
| 2.11 | UUG-AUGG |
| 2.12 | GUU-AUGG |
| 2.13 | GGG-AUGA |
| 2.14 | UGG-AUGG |
| 2.15 | UUU-AUGG |
| 2.16 | ACC-ACGG |
| 2.17 | UUU-UUUG |
| 2.18 | ACC.AUGGGUUGAUUUUUUUUU-AUGG |
| 2.19 | ACC.AUGGGUUGAUUUUUUUU-AUGG |
| 2.20 | ACC.AUGGGUUGAUUUUUUU-AUGG |
| 2.21 | ACC.AUGGGUUGAUUUUUU-AUGG |
| 2.22 | ACC.AUGGGUUGAUUUUU-AUGG |
| 2.23 | ACC.AUGGGUUGAUUUU-AUGG |
| 2.24 | ACC.AUGGGUUGAUUU-AUGG |
| 2.25 | ACCAUGGGUUGAUU-AUGG |
| 2.26 | ACC.AUGGGUUGAU-AUGG |
| 2.27 | ACC.AUGGGUUGA-AUGG |
| 2.28 | ACC.AUGGGUUG-AUGG |
| 2.29 | ACC.AUGGGUU-AUGG |
| 2.30 | ACC.AUGGG-AUGA |
| 2.31 | ACC.AUGG-AUGGGUGA |
| 2.36 | UUU.AUGGGUUGAUUUUU-AUGG |
| 2.46 | ACC.AUGGGUUGAUUACC-AUGG |
| 2.47 | UUU.AUGGGUUGAUUACC-AUGG |
| 2.48 | ACC.AUGGGUUGAUUACC-ACGG |
| 2.49 | UUU.AUGGGUUGAUUACC-ACGG |
| 2.50 | ACC.AUGGGUUGAUUUUU-ACGG |
| 2.51 | UUU.AUGGGUUGAUUUUU-ACGG |
| 2.52 | ACC.AUGGGUUGA-UUUG |
| 3.1 | GCCACC-AUGG |
| 3.2 | CAG-AUGG |
| 3.3 | CGA-AUGG |
| 3.4 | GAA-AUGG |
| 3.5 | UAA-AUGG |
| 3.6 | UAG-AUGG |
| 3.7 | UGC-AUGG |
| 3.8 | UUU.AUGGGUUGAUUAUU-AUGG |
| 3.9 | UUU.AUGGGUUGAUUCAG-AUGG |
| 3.10 | UUU.AUGGGUUGAUUCGA-AUGG |
| 3.11 | UUU.AUGGGUUGAUUGAA-AUGG |
| 3.12 | UUU.AUGGGUUGAUUGGG-AUGG |
| 3.13 | UUU.AUGGGUUGAUUUAA-AUGG |
| 3.14 | UUU.AUGGGUUGAUUUAG-AUGG |
| 3.15 | UUU.AUGGGUUGAUUUCC-AUGG |
| 3.16 | UUU.AUGGGUUGAUUUGA-AUGG |
| 3.17 | UUU.AUGGGUUGAUUUGC-AUGG |
| 3.18 | UUU.AUGGGUUGAUUUUG-AUGG |
| 3.19 | AUU.AUGGGUUGAUUUUU-AUGG |
| 3.20 | GGG.AUGGGUUGAUUUUU-AUGG |
| 3.21 | UCC.AUGGGUUGAUUUUU-AUGG |
| 3.22 | UGA.AUGGGUUGAUUUUU-AUGG |
| 3.23 | UUG.AUGGGUUGAUUUUU-AUGG |
| 3.24 | ACC.AUGGGUUGAUUUGG-AUGG |
| 4.1 | CGG-AUGG |
| 4.2 | CUU-AUGG |
| 4.3 | GGC-AUGG |
| 4.4 | GGG-AUGG |
| 4.5 | UCG-AUGG |
| 4.6 | UGA.AUGGGUUGAUUACC-AUGG |
| 4.7 | UGG.AUGGGUUGAUUACC-AUGG |
| 4.8 | UGC.AUGGGUUGAUUACC-AUGG |
| 4.9 | UGA.AUGGGUUGAUUUCC-AUGG |
| 4.10 | UGG.AUGGGUUGAUUUCC-AUGG |

*Dash precedes start codon of mEGFP & base at position +1; period precedes start codon of upstream ORF (underlined).

### EXAMPLE 2

### Derivation of a Model Describing uORF Suppression of Expression from a GOI ORF

This model is based on the assumption that partial or “leaky” initiation at the uORF allows a fraction of ribosomes to reach and translate a downstream, gene of interest (GOI) ORF. We define our variables and parameters as follows:

T=Translation initiation rate

R=ribosomal flux

P=probability of initiation when the ribosome encounters a TIS sequence

S=Strength of TIS based on observed GFP expression level without an uORF

k=proportionality constant relating TIS strength to initiation probability

X=relative expression level

Items associated with the uORF and GOI ORF are represented with subscripts u and G, respectively.

The translation initiation rate depends on the flux of ribosomes and the probability of translation initiation.

T=PR

Because no translation has occurred upstream of the uORF, the initial ribosome flux is equal to the flux that reaches the uORF, Rᵤ. Then at the uORF, a fraction of ribosomes will initiate translation according to

Tᵤ=PᵤRᵤ

The fraction of ribosomes that does not initiate and continues to the GOI ORF can then be described as

R_{G}=(1−Pᵤ)Rᵤ

and the translation initiation rate of the GOI is then described by the following:

T_{G}=P_{G}R_{G}

T_{G}=P_{G}(1−Pᵤ)Rᵤ

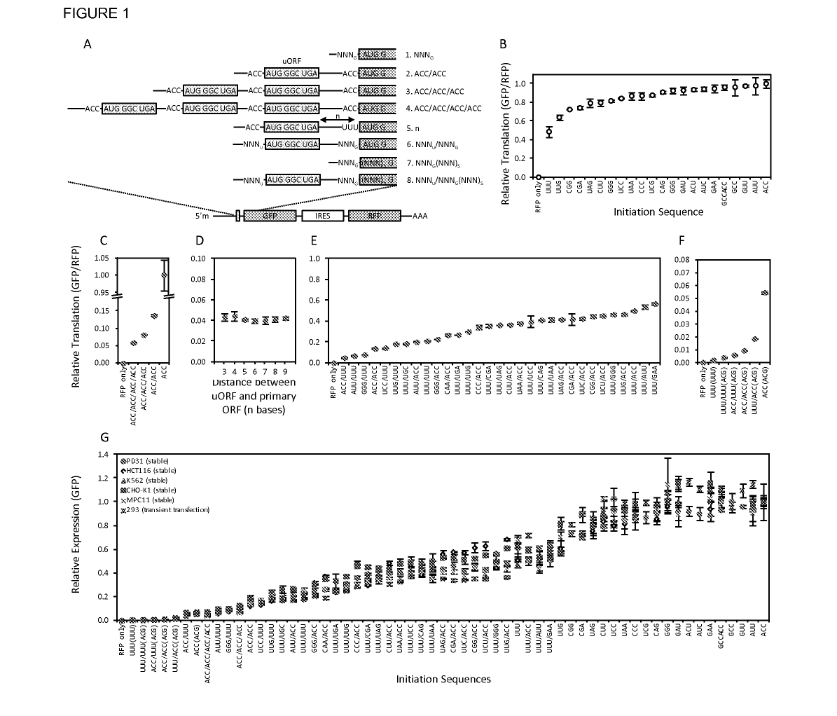

We make the assumption that the probability of initiation is proportional to the relative GFP/RFP expression levels determined from our expression constructs where we varied the TIS sequences but did not employ uORFs (FIG. 1B). We designate the GFP/RFP expression levels as measurements of TIS strength, S. It follows then that

Pᵤ=kSᵤ and P_{G}=kS_{G}

T_{G}=kS_{G}(1−kSᵤ)Rᵤ

T_{G} here is an absolute level of translation initiation with units of initiation events per time. Yet our experimental measurements of GFP expression have relative expression units, where we have divided our fluorescence intensity levels by the level of GFP fluorescence intensity produced by the reference TIS ACCAUGG (without any uORF). To generate a model equation that allows us to directly fit our experimental data we also normalize to a reference,

T_{ref}=kS_{ref}R_{ref}

X_{G}=T_{G}/T_{ref}

R_{ref} and Rᵤ are identical because both are the ribosomal flux before ribosomes reach any open reading frame, allowing us to eliminate the ribosomal flux terms when solving for relative expression. Furthermore, in our case we set S_{ref} to 1 for convenience thus,

X_{G}=(1−kSᵤ)S_{G}

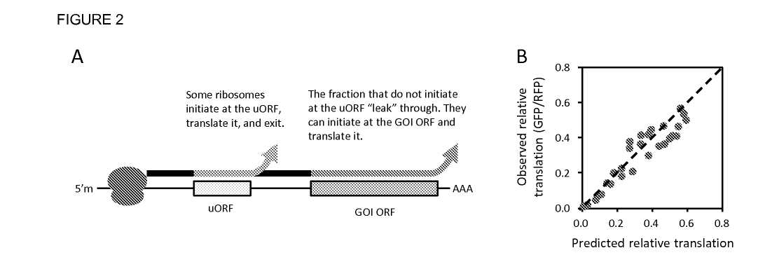

Because our mathematical description of expression is based on a probabilistic decision-making mechanism, after determining the value of k from our experimental data (FIG. 1B and FIG. 2C), we can also approximate a probability of initiation by a ribosome for each TIS sequence (P_{TIS}) based on experimentally evaluated expression levels without any uORF (X_{TIS}).

P_{TIS}=kX_{TIS}

Other embodiments of the invention will be apparent to those skilled in the art from a consideration of the specification or practice of the invention disclosed herein. It is intended that the specification and Examples be considered as exemplary only, with the true scope of the invention being indicated by the following claims.

What is claimed is:

1. A library of DNA sequences for regulating expression of a gene of interest, wherein each of the DNA sequences comprises:
a promoter;
a first translation initiation sequence operably linked to a first upstream regulatory open reading frame (ORF) of from two to 10 codons in length, which is from 2 to 10 nucleotides distant from the initiation codon of the gene of interest;
a second translation initiation sequence operably linked to the gene of interest; and wherein the DNA sequences in the library provide for a range of expression levels of at least 100-fold.

2. The library of claim 1, wherein the range of expression levels is at least 200-fold.

3. The library of claim 1, wherein the promoter is a strong eukaryotic promoter.

4. The library of claim 1, wherein the individual DNA sequences are varied in one or both of the first and the second translation initiation sequences.

5. The library of claim 1, wherein the DNA sequences are comprised with a construct for expression.

6. The library of claim 5, wherein the constructs are introduced into eukaryotic cells for expression.

7. The library of claim 1, wherein one or more of said DNA sequences comprises a third translation initiation sequence operably linked to a second upstream regulatory open reading frame of from two to 10 codons in length, which is from 2 to 10 nucleotides upstream from the first upstream regulatory open reading frame.

8. A method of screening to select a regulatory sequence that provides a desired for level of expression of a gene of interest, the method comprising:
operably linked a gene of interest to a second translation initiation sequence of the library of claim 1;
introducing the library into a cell of interest for expression;
determining the level of expression from individual members of the library.

9. An expression construct selected by the method of claim 8.

10. A DNA sequence for regulating expression of a gene of interest, comprising: a promoter; a first translation initiation sequence operably linked to an upstream regulatory open reading frame (ORF) of from two to 10 codons in length, which is from 2 to 10 nucleotides distant from the initiation codon of the gene of interest; a second translation initiation sequence operably linked to the gene of interest.

11. The DNA sequence of claim 10, further comprising a third translation initiation sequence operably linked to a second upstream regulatory open reading frame of from two to 10 codons in length, which is from 2 to 10 nucleotides upstream from the first upstream regulatory open reading frame.

12. An expression vector comprising the DNA sequence of claim 10 or claim 11, operably linked to a gene of interest.

13. A eukaryotic host cell comprising the expression vector of claim 12.


SEQUENCE LISTING (56 sequences)

SEQ ID NO: 1 - RNA, 13 nt, Artificial sequence
  Synthetic nucleic acid sequence
gccgccrccaugg

SEQ ID NO: 2 - DNA, 58 bp, Artificial sequence
  Synthetic nucleic acid sequence
cggaattggccgccctagatgcatgcttattcgaacttgtacagctcgtccatgccga

SEQ ID NO: 3 - RNA, 10 nt, Artificial sequence
  Synthetic nucleic acid sequence
gccaccaugg

SEQ ID NO: 4 - RNA, 19 nt, Artificial sequence
  Synthetic nucleic acid sequence
accauguuuugauuuauga

SEQ ID NO: 5 - RNA, 17 nt, Artificial sequence
  Synthetic nucleic acid sequence
accauguuuugauauga

SEQ ID NO: 6 - RNA, 16 nt, Artificial sequence
  Synthetic nucleic acid sequence
accauguuuugaauga

SEQ ID NO: 7 - RNA, 15 nt, Artificial sequence
  Synthetic nucleic acid sequence
accauguuuugauga

SEQ ID NO: 8 - RNA, 14 nt, Artificial sequence
  Synthetic nucleic acid sequence
accauguuuuauga

SEQ ID NO: 9 - RNA, 12 nt, Artificial sequence
  Synthetic nucleic acid sequence
accauguuauga

SEQ ID NO: 10 - RNA, 15 nt, Artificial sequence
  Synthetic nucleic acid sequence
accauguuuugacga

SEQ ID NO: 11 - RNA, 25 nt, Artificial sequence
  Synthetic nucleic acid sequence
accauggguugauuuuuuuuuaugg

SEQ ID NO: 12 - RNA, 24 nt, Artificial sequence
  Synthetic nucleic acid sequence
accauggguugauuuuuuuuaugg

SEQ ID NO: 13 - RNA, 23 nt, Artificial sequence
  Synthetic nucleic acid sequence
accauggguugauuuuuuuaugg

SEQ ID NO: 14 - RNA, 22 nt, Artificial sequence
  Synthetic nucleic acid sequence
accauggguugauuuuuuaugg

SEQ ID NO: 15 - RNA, 21 nt, Artificial sequence
  Synthetic nucleic acid sequence
accauggguugauuuuuaugg

SEQ ID NO: 16 - RNA, 20 nt, Artificial sequence
  Synthetic nucleic acid sequence
accauggguugauuuuaugg

SEQ ID NO: 17 - RNA, 19 nt, Artificial sequence
  Synthetic nucleic acid sequence
accauggguugauuuaugg

SEQ ID NO: 18 - RNA, 18 nt, Artificial sequence
  Synthetic nucleic acid sequence
accauggguugauuaugg

SEQ ID NO: 19 - RNA, 17 nt, Artificial sequence
  Synthetic nucleic acid sequence
accauggguugauaugg

SEQ ID NO: 20 - RNA, 16 nt, Artificial sequence
  Synthetic nucleic acid sequence
accauggguugaaugg

SEQ ID NO: 21 - RNA, 15 nt, Artificial sequence
  Synthetic nucleic acid sequence
accauggguugaugg

SEQ ID NO: 22 - RNA, 14 nt, Artificial sequence
  Synthetic nucleic acid sequence
accauggguuaugg

SEQ ID NO: 23 - RNA, 12 nt, Artificial sequence
  Synthetic nucleic acid sequence
accaugggauga

SEQ ID NO: 24 - RNA, 15 nt, Artificial sequence
  Synthetic nucleic acid sequence
accauggauggguga

SEQ ID NO: 25 - RNA, 21 nt, Artificial sequence
  Synthetic nucleic acid sequence
uuuauggguugauuuuuaugg

SEQ ID NO: 26 - RNA, 21 nt, Artificial sequence
  Synthetic nucleic acid sequence
accauggguugauuaccaugg

SEQ ID NO: 27 - RNA, 21 nt, Artificial sequence
  Synthetic nucleic acid sequence
uuuauggguugauuaccaugg

SEQ ID NO: 28 - RNA, 21 nt, Artificial sequence
  Synthetic nucleic acid sequence
accauggguugauuaccacgg

SEQ ID NO: 29 - RNA, 21 nt, Artificial sequence
  Synthetic nucleic acid sequence
uuuauggguugauuaccacgg

SEQ ID NO: 30 - RNA, 21 nt, Artificial sequence
  Synthetic nucleic acid sequence
accauggguugauuuuuacgg

SEQ ID NO: 31 - RNA, 21 nt, Artificial sequence
  Synthetic nucleic acid sequence
uuuauggguugauuuuuacgg

SEQ ID NO: 32 - RNA, 16 nt, Artificial sequence
  Synthetic nucleic acid sequence
accauggguugauuug

SEQ ID NO: 33 - RNA, 21 nt, Artificial sequence
  Synthetic nucleic acid sequence
uuuauggguugauuauuaugg

SEQ ID NO: 34 - RNA, 21 nt, Artificial sequence
  Synthetic nucleic acid sequence
uuuauggguugauucagaugg

SEQ ID NO: 35 - RNA, 21 nt, Artificial sequence
  Synthetic nucleic acid sequence
uuuauggguugauucgaaugg

SEQ ID NO: 36 - RNA, 21 nt, Artificial sequence
  Synthetic nucleic acid sequence
uuuauggguugauugaaaugg

SEQ ID NO: 37 - RNA, 21 nt, Artificial sequence
  Synthetic nucleic acid sequence
uuuauggguugauugggaugg

SEQ ID NO: 38 - RNA, 21 nt, Artificial sequence
  Synthetic nucleic acid sequence
uuuauggguugauuuaaaugg

SEQ ID NO: 39 - RNA, 21 nt, Artificial sequence
  Synthetic nucleic acid sequence
uuuauggguugauuuagaugg

SEQ ID NO: 40 - RNA, 21 nt, Artificial sequence
  Synthetic nucleic acid sequence
uuuauggguugauuuccaugg

SEQ ID NO: 41 - RNA, 21 nt, Artificial sequence
  Synthetic nucleic acid sequence
uuuauggguugauuugaaugg

SEQ ID NO: 42 - RNA, 21 nt, Artificial sequence
  Synthetic nucleic acid sequence
uuuauggguugauuugcaugg

SEQ ID NO: 43 - RNA, 21 nt, Artificial sequence
  Synthetic nucleic acid sequence
uuuauggguugauuuugaugg

SEQ ID NO: 44 - RNA, 21 nt, Artificial sequence
  Synthetic nucleic acid sequence
auuauggguugauuuuuaugg

SEQ ID NO: 45 - RNA, 21 nt, Artificial sequence
  Synthetic nucleic acid sequence
gggauggguugauuuuuaugg

SEQ ID NO: 46 - RNA, 21 nt, Artificial sequence
  Synthetic nucleic acid sequence
uccauggguugauuuuuaugg

SEQ ID NO: 47 - RNA, 21 nt, Artificial sequence
  Synthetic nucleic acid sequence
ugaauggguugauuuuuaugg

SEQ ID NO: 48 - RNA, 21 nt, Artificial sequence
  Synthetic nucleic acid sequence
uugauggguugauuuuuaugg

SEQ ID NO: 49 - RNA, 21 nt, Artificial sequence
  Synthetic nucleic acid sequence
accauggguugauuuggaugg

SEQ ID NO: 50 - RNA, 21 nt, Artificial sequence
  Synthetic nucleic acid sequence
ugaauggguugauuaccaugg

SEQ ID NO: 51 - RNA, 21 nt, Artificial sequence
  Synthetic nucleic acid sequence
uggauggguugauuaccaugg

SEQ ID NO: 52 - RNA, 21 nt, Artificial sequence
  Synthetic nucleic acid sequence
ugcauggguugauuaccaugg

SEQ ID NO: 53 - RNA, 21 nt, Artificial sequence
  Synthetic nucleic acid sequence
ugaauggguugauuuccaugg

SEQ ID NO: 54 - RNA, 21 nt, Artificial sequence
  Synthetic nucleic acid sequence
uggauggguugauuuccaugg

SEQ ID NO: 55 - RNA, 12 nt, Artificial sequence
  Synthetic nucleic acid sequence
accaugggcuga

SEQ ID NO: 56 - RNA, 12 nt, Artificial sequence
  Synthetic nucleic acid sequence
nnnaugggcuga